(19) United States
(12) Patent Application Publication
(10) Pub. No.: US 2026/0345942 A1
(43) Pub. Date: Nov. 5, 2026

(54) PARAMETER MEASUREMENT FOR INFANT RESUSCITATION DEVICES

(71) Applicant: GE Precision Healthcare LLC, Waukesha, WI (US)

(72) Inventors: Helge B. Klockow, Wauwatosa, WI (US); Steven M. Falk, Baltimore, MD (US)

(73) Assignee: GE Precision Healthcare LLC, Waukesha, WI (US)

(21) Appl. No.: 19/195,867

(22) Filed: May 1, 2025

(51) Int. Cl. A61M 16/12; A61G 11/00; A61M 16/00; A61M 16/10

(52) U.S. Cl. CPC A61M 16/125; A61M 16/0051; A61M 16/024; A61M 16/101; A61G 11/00; A61M 2016/0027; A61M 2016/1025; A61M 2205/581; A61M 2205/583; A61M 2205/702; A61M 2240/00

(57) ABSTRACT

An infant resuscitation device includes a blender control device, blender device, oxygen sensor, and display device. The blender control device selects an oxygen concentration of an air supply that the infant resuscitation device provides at an exhaust port. Additionally, the blender device generates the air supply by combining medical grade air and medical grade oxygen based on a selection of the oxygen concentration by the blender control device. Further, the oxygen sensor is disposed within a portion of a respiratory circuit of the infant resuscitation device, and generates a measure by measuring an oxygen concentration of the air supply generated by the blender device. Additionally, the display device, is disposed on an external portion of the infant resuscitation device, receives the measure from the oxygen sensor, and displays the measure of the oxygen concentration.

### BACKGROUND

The present disclosure generally relates to parameter measurement, and more specifically to parameter measurement for infant resuscitation devices.

Infant care devices may be useful for neonates and prematurely born infants who are not physiologically developed enough to be able to survive without medical attention. A frequently used medical aid for such infants is the infant resuscitation device. The infant resuscitation device provides the neonate an air supply under pressure with a specific oxygen concentration. In this way, the infant resuscitation device may enable the neonate to breathe life-sustaining air.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

An infant resuscitation device includes a blender control device, blender device, oxygen sensor, and display device. The blender control device selects an oxygen concentration of an air supply that the infant resuscitation device provides at an exhaust port. Additionally, the blender device generates the air supply by combining medical grade air and medical grade oxygen based on a selection of the oxygen concentration by the blender control device. Further, the oxygen sensor is disposed within a portion of a respiratory circuit of the infant resuscitation device, and generates a measure by measuring an oxygen concentration of the air supply generated by the blender device. Additionally, the display device, is disposed on an external portion of the infant resuscitation device, receives the measure from the oxygen sensor, and displays the measure of the oxygen concentration.

In some embodiments, the first portion of the respiratory circuit is configured to receive the air supply before a manometer of the infant resuscitation device receives the air supply.

In some embodiments, the first oxygen sensor is disposed between the blender device and the exhaust port.

In some embodiments, the first oxygen sensor is disposed between the blender device and the manometer.

In some embodiments, the infant resuscitation device includes a second oxygen sensor that is disposed within a second portion of the respiratory circuit of the infant resuscitation device, and is configured to generate a second measure by measuring the oxygen concentration of the air supply generated by the blender device.

In some embodiments, the second portion of the respiratory circuit is configured to receive the air supply from a manometer of the infant resuscitation device.

In some embodiments, the infant resuscitation device includes a circuit configured to compare the first measure to the second measure, determine if the first measure is within a predetermined threshold of the second measure, and provide an indicator for the first display to display an error message.

In some embodiments, the infant resuscitation device includes a second display device that is disposed on a second external portion of the infant resuscitation device, and is configured to receive the second measure from the second oxygen sensor, and display the second measure of the oxygen concentration.

In some embodiments, the infant resuscitation device includes pneumatic components configured to receive the medical grade air and the medical grade oxygen, generate the air supply by blending the medical grade air and the medical grade oxygen, and deliver the air supply under pressure at the exhaust port.

A system includes a processor and a memory. The memory includes instructions that cause the processor to receive a set point of oxygen concentration for an infant resuscitation device, receive, from a first oxygen sensor, a first measure of oxygen concentration of air generated by a blender of the infant resuscitation device, present the first measure of oxygen concentration on an output device, compare the first measure to the set point, determine that the first measure exceeds a predetermined threshold of the set point based on the comparison, and generate, in response to the determination, an alert that the oxygen concentration is not within the predetermined threshold of the set point.

In some embodiments, the system includes the output device, and the instructions cause the output device to perform at least one function selected from a group consisting of displaying the alert, displaying the first measure, generating an audio signal representing the alert, generating an audio message comprising the first measure.

In some embodiments, the system includes a blender control device configured to select the set point of oxygen concentration of an air supply that the apparatus provides at an exhaust port.

In some embodiments, the system includes the infant resuscitation device, and the infant resuscitation device includes a blender device configured to generate the air supply by combining medical grade air and medical grade oxygen based on the set point, and a first oxygen sensor that is disposed within a first portion of a respiratory circuit of the infant resuscitation device, and is configured to generate the first measure by measuring an oxygen concentration of the air supply generated by the blender device.

In some embodiments, the first portion of the respiratory circuit is configured to receive the air supply before a manometer of the infant resuscitation device receives the air supply.

In some embodiments, the first oxygen sensor is disposed between the blender device and the exhaust port.

In some embodiments, the first oxygen sensor is disposed between the blender device and the manometer.

In some embodiments, the infant resuscitation device includes a second oxygen sensor that is disposed within a second portion of the respiratory circuit of the infant resuscitation device, and is configured to generate a second measure by measuring the oxygen concentration of the air supply generated by the blender device.

In some embodiments, the second portion of the respiratory circuit is configured to receive the air supply from a manometer of the infant resuscitation device.

In some embodiments, the second oxygen sensor is disposed between the first oxygen sensor and the exhaust port.

In some embodiments, the infant resuscitation device includes pneumatic components configured to receive the medical grade air and the medical grade oxygen, generate the air supply by blending the medical grade air and the medical grade oxygen, and deliver the air supply under pressure at the exhaust port.

Various other features, objects, and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a perspective view of an example system having parameter measurement for infant resuscitation devices, according to some embodiments of the present disclosure.

FIG. 2A is a perspective view of an example system having parameter measurement for infant resuscitation devices, according to some embodiments of the present disclosure.

FIG. 2B is a perspective view of an example infant resuscitation device having parameter measurement, according to some embodiments of the present disclosure.

FIG. 3A is a perspective view of an example system having parameter measurement for infant resuscitation devices, according to some embodiments of the present disclosure.

FIG. 3B is a diagram of an example circuit infant resuscitation device having parameter measurement, according to some embodiments of the present disclosure.

FIG. 4 is a diagram of an example respiratory circuit of an infant resuscitation device having parameter measurement, according to some embodiments of the present disclosure.

FIG. 5 is a diagram of graphs representing respiratory pressure, tidal volume, and mask volume of an infant resuscitation device having parameter measurement, according to some embodiments of the present disclosure.

FIG. 6 is an example resuscitation measurement manager, according to some embodiments of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally or alternatively, embodiments may be arranged in a different orientation such that “top” and “bottom” features are arranged horizontally relative to each other, for example in a “left-to-right” orientation.

The use herein of the terms “including,” “including,” or “having,” and variations thereof, is meant to encompass the elements listed thereafter and equivalents thereof, as well as additional elements. Embodiments recited as “including,” “including,” or “having” certain elements are also contemplated as “consisting essentially of” and “consisting of” those certain elements.

The inventors have recognized a problem with current pneumatic resuscitation devices for infants, which is that there is no way to determine the concentration of oxygen that the pneumatic resuscitation device is supplying the infant. For example, pneumatic resuscitation devices for infants include controls for multiple parameters such as, the pressure flow rate, and the oxygen concentration. Additionally, these devices may include manometers that provide a visual display of the pressure. Further, these devices may also include alarms that trigger when an input pressure line drops below a predetermined threshold. Additionally, a clinician or other device operator may use a blender control knob to set the oxygen concentration to a specific percentage between 21 and 100 percent. The blender may be an internal component of the resuscitation device that combines medical grade air an oxygen according to the oxygen concentration setting of the blender control knob. However, for various reasons, the blender may not work occasionally, e.g., it may not generate air with the specified oxygen concentration. Accordingly, health care facilities may schedule service intervals to ensure that the blender is working. If there is a problem with the blender, a technician may fix it. In place of the resuscitation device with the problematic blender, a clinician that is administering resuscitation care to an infant may use a different device. However, these service intervals may not ensure that the blender is working while in operation. Rather, the service provided at the service interval may ensure the blender is working on one day. However, the blender may break between service intervals. As such, determining that the blender is not working during operation may involve the pediatrician or neonatologist observing the infant receiving resuscitation from the device and noticing a problem resulting from the infant receiving insufficient oxygen, e.g., the baby's skin takes on a bluish tone. In such a scenario, it may be possible to put the infant on a different device. However, the infant may suffer harm from the lack of oxygen. Additionally, in cases where the device is providing too high a concentration of oxygen, the infant may also suffer harm, e.g., too high an oxygen exposure to an infants eyes may harm their sight, a condition which may not be as easy to detect as a lack of oxygen.

Through significant research and experimentation, the inventors developed the disclosed pneumatic resuscitation devices for infants with oxygen concentration indicator, which provides improved patient outcomes by keeping health care professionals informed about the oxygen concentration that the pneumatic resuscitation device is providing an infant. These systems can further be configured to provide an alarm when the oxygen concentration falls below a setpoint and/or indicator that the oxygen concentration is within a set point range.

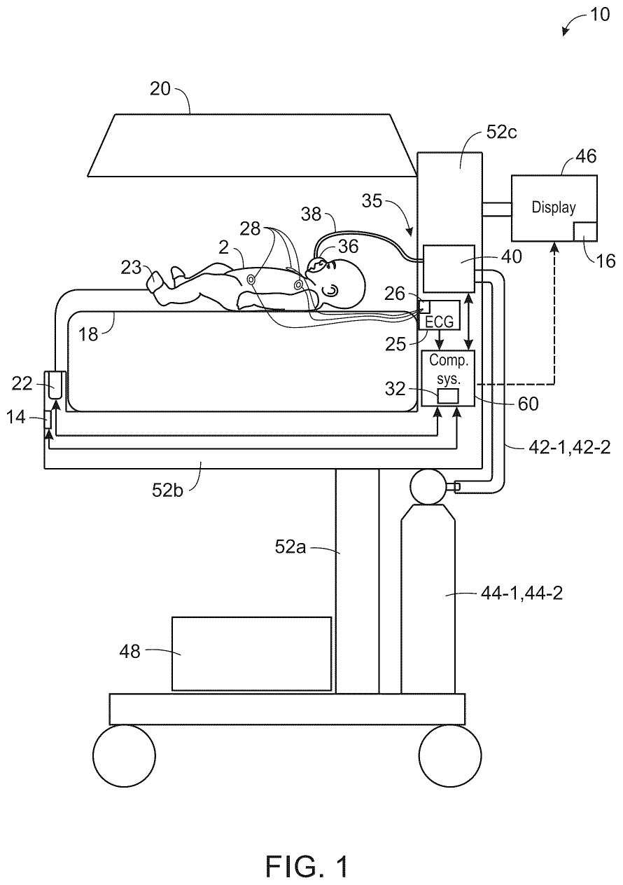

FIG. 1 is a perspective view of an example infant warming system 10 having parameter measurement for infant resuscitation devices, according to some embodiments of the present disclosure. The example infant warming system 10 is a radiant warmer having a radiant heater 20 that directs infrared energy towards an infant 2 in order to provide a heated environment. According to some embodiments of the present disclosure, the infant warming system 10 may include an incubator system, alone or in combination with the radiant heater 20, where a canopy is provided over the platform 18 supporting the infant 2 to form a micro environmental chamber providing a controlled and isolated environment. However, not having a canopy, the infant warming system 10 may provide unimpeded access to the infant 2 for purposes of providing resuscitative and ventilation care within the first few minutes of birth. The position of the infant 2 in FIG. 1 is not intended to be indicative or representative of actual or appropriate positioning of an infant 2 for resuscitation. When performing resuscitative and ventilation care to the infant 2 in the first few minutes of birth, the infant 2 may be placed with their head proximal to the bottom, or foot side, of the platform 18.

The example warming system 10 includes a frame structure 52, which provides structural support for, and houses, the various aspects of the warming system 10. The frame structure 52, for example, may include structural support elements supporting the weight of the system components, as well as exterior or casing elements that enclose and protect the system components and/or provide an attractive facade. Further, in this example, the frame structure 52 includes a base portion 52a (which may support and/or house various aspects of the system, such as a battery 48 and/or gas supply tanks 44-1, 44-2 having medical grade gases), a platform support structure 52b portion that supports the platform 18 on which the infant 2 lays, and a vertical panel structure 52c (which may support various user interface and control systems, for example, as well as systems for providing therapy and care to the infant 2, such as a resuscitation device 40, an ECG monitor 25, the radiant heater 20, an SpO₂ monitor 22, and the like). According to some embodiments of the present disclosure, the system 10 may include any of various additional system elements, which may be arranged on the frame structure 52 in various ways not exemplified in the infant warming system 10.

Devices and systems for monitoring and providing resuscitation and other therapies to the infant 2 are incorporated into the infant warming system 10. In this example, the infant warming system 10 includes an ECG monitor 25 housed on the frame structure 52. The ECG monitor 25 may receive cardiac potentials via ECG electrodes 28 that are connected to the infant 2 at the right arm RA, left arm LA, and left leg LL positions. Alternatively, the ECG monitor 25 may receive cardiac potentials from any number of two or more electrodes connected to the infant 2 in any of various electrode arrangements. The ECG electrodes 28 are connected to the ECG monitor 25 via an ECG connection port 26 configured to receive connectors of the two or more ECG electrodes 28.

The ECG connection port 26 may be positioned on and provided at a portion of the frame structure 52 that is proximal to a head of the platform 18. In various embodiments, the ECG connection port 26 may take any of various forms. The ECG electrode connection port provides a physical contact point for connection with each electrode 28, which may be bundled together in a single connector that mates with ECG connection port 26, or each electrode 28 may have a separate connector that mates with and is separately received by the ECG connection port 26. In still other embodiments, the ECG electrodes 28 may be wireless electrodes, and in such an embodiment the ECG connection port 26 may include one or more wireless receiver transmitters in communication with two or more ECG wireless electrodes.

The infant warming system 10 may further include a pulse oximeter device 22, including an SpO₂ sensor 23 attachable to the infant 2 to measure, or provide an estimate of, the oxygen saturation (SpO₂) value of the infant's red blood cells. The pulse oximeter device 22 may be incorporated into and provided at any location on the frame structure 52. In some embodiments of the present disclosure, the pulse oximeter 22 is provided on the platform support structure 52b, and specifically at a location proximal to a foot side of the platform 18. Alternatively, the pulse oximeter and/or a connection for the sensor 23 may be provided at any location on the frame structure 52, such as on the vertical panel structure 52c. For example, the vertical panel structure 52c may include an SpO₂ sensor port (not shown). Alternatively, the SpO₂ sensor 23 may be a wireless device configured to wirelessly transmit SpO₂ measurement values to a receiver transmitter incorporated within the system 10.

In this example, the ECG monitor 25 and the pulse oximeter device 22 are depicted as separate devices that calculate respective monitoring values for the infant 2 and transmit such values to a computing system 60. The computing system 60 may include a processor and a memory (not shown). The memory may include instructions that are executable by the processor to perform resuscitation techniques as described herein. Further, communication with the computing system 60 may be provided by any wired or wireless means. According to some embodiments of the present disclosure, some or all of the physical circuitry and/or software including the pulse oximeter 22 and/or the ECG monitor 25 may be incorporated within the computing system 60. In general, the control system for the warming system 10 may include several separate computing systems, or control sub-systems, in the ECG monitor 25, the pulse oximeter device 22, the resuscitation device 40, the computing system 60, and/or various other control sub-systems for controlling various aspects of the system 10, such as a display control sub-system, a speaker control sub-system, an auditory processing system associated with a microphone 14, and the like. In various embodiments, all such sub-systems may be provided on separate hardware systems, or any or all of them may be combined together in a single set of hardware and software, and together may be generally referred to herein as the control system for the warming system 10.

The warming system 10 may further include devices and systems for providing ventilation support for the infant 2. For example, a breathing circuit 35 for providing gas to the infant 2 includes a resuscitation device 40, such as a positive pressure ventilation (PPV) device, or a positive end expiratory pressure (PEEP) device (or a ventilator device providing all three respiratory therapies). Further, the resuscitation device 40 receives a gas supply from supply lines 42-1, 42-2 respectively connected to gas supply tanks 44-1, 44-2 supported on the base structure 52a. The gas supply tank 44-1 may include a supply of medical grade oxygen (O₂). Additionally, the gas supply tank 44-2 may include a supply of medical grade air. According to some embodiments of the present disclosure, the resuscitation device 40 may be a pneumatic resuscitation device.

The resuscitation device 40 regulates the gas supply to provide resuscitative and/or respiratory assistance to the infant 2. In this example, the resuscitation device 40 connects to a breathing tube 38 supplying gas to the infant 2 through a mask 36 applied over the infant's nose and mouth. According to some embodiments of the present disclosure, the mask 36 may be attached to a T-piece, or a bag and mask system (not shown). The T-piece may have an opening that a clinician or other healthcare provider covers while the resuscitation device 40 inflates the patients lungs. Conversely, the healthcare provider may leave the opening uncovered while the patient exhales. In other embodiments, gas may be delivered to the patient via another device, such as an endotracheal tube, a laryngeal mask, nasal cannula, or the like, and any such gas delivery element or system may connect to the breathing tube 38 in order to deliver gas from the gas supply tanks 44-1, 44-2, to the patient.

The infant warming system 10 may include a battery 48 to power the various systems and devices thereon. The battery 48 may be positioned, for example, on the base structure 52a, such as at a location that is accessible in order to recharge or replace that battery 48. Alternatively, or additionally, the infant radiant heater 20 may receive power from being plugged into an outlet (not shown) that is connected to an AC power grid for the health care facility. The charging status of the battery may be monitored by a power control module, such as may be provided separately from and in communication with, or otherwise incorporated into, the computing system 60. The computing system 60 may provide a battery status notification, such as on the display device 46.

The warming system 10 may include various user interface devices for controlling various aspects of the system. Such user interface devices may include a display device 46 controllable to provide physiological information about the infant 2 and/or the status of various aspects of the system. For example, the display 46 may be controllable by the computing system 60 to display a heart rate, SpO₂, a pulse rate, a temperature, or any other physiological information measured from the patient. Additionally, the display device 46 may be controlled to display information regarding the heater system 20, resuscitation device 40, ECG monitor 25, or pulse oximeter device 22, such as the mode of operation or other pertinent regarding those systems and devices. In certain embodiments, the display device 46 may be a touch screen capable of providing user control inputs through which a clinician can control the various systems and devices including the infant warming system 10. According to some embodiments of the present disclosure, a clinician may use the touch screen (or other input device) to control the oxygen concentration that the resuscitation system 40 provides to the infant. The concentration of oxygen selected using such input devices is referred to herein as the setpoint of oxygen concentration.

According to some embodiments of the present disclosure, the resuscitation device 40 may include a control to regulate the setpoint of the oxygen concentration in the gases provided for the infant 2. Additionally, the resuscitation device 40 may include one or more displays (not shown) connected with one or more corresponding oxygen sensors (not shown) within the resuscitation device 40. The oxygen sensors may automatically determine the concentration of oxygen in the air that the resuscitation device 40 is providing to the infant, and provide the determined concentration to the displays. In this way, the resuscitation device 40 may display the oxygen concentration detected by the oxygen sensors. Accordingly, a healthcare provider may observe the display(s) to confirm if the oxygen concentration provided by the resuscitation device 40 is within a predetermined threshold of the oxygen concentration set by the controls of the resuscitation device 40.

The infant warming system 10 may further include a microphone 14 configured to detect voice inputs from a clinician, such as during operation of the system in resuscitation mode as described herein. The system may further include a speaker 16, which may be incorporated into the display device 46 or elsewhere on the infant warming system, that produces audible alerts, alarms, instructions, or the like to facilitate care of the infant 2.

The computing system 60 may include a resuscitation manager 32 having executable software instructions for system control that provide a system for resuscitative support within the first few minutes after the infant's birth. Various possible control features and aspects provided by the resuscitation manager 32 are discussed herein. More specifically, the resuscitation manager 32 may provide indicators that describe the operation of the resuscitation device 40 and/or breathing circuit 35. For example, the resuscitation manager 32 may determine the tidal volume of gases that the resuscitation device 40 is providing to the infant 2. Additionally, the resuscitation manager 32 may determine the amount of any mask leak. The mask 36 may not provide a complete seal around the infant's mouth and nose, and thus, some of the gas provided for the infant 2 may leak through this incomplete seal. Accordingly, the mask leak may represent the amount of gas provided by the resuscitation device 40 that is leaking out of the mask 36, and thus not being provided to the infant. Further, the resuscitation manager 32 may display the tidal volume and/or mask leak for display on the display device 46. Additionally, the resuscitation manager 32 may determine and provide for display on the display device 46, an oxygen concentration in the gas that the resuscitation device 40 is delivering to the infant 2. Additionally, the resuscitation manager 32 may compare the oxygen concentration detected by the oxygen sensors to the setpoint of oxygen concentration. As stated previously, if the oxygen concentration provided to the infant 2 is too low, or too high, the infant 2 may suffer harm. Accordingly, if the oxygen concentration is not within a predetermined threshold of the setpoint, the resuscitation manager 32 may generate an alert, e.g., a visual or audio alarm to attract the attention of a clinician or other caregiver to remedy the discrepancy.

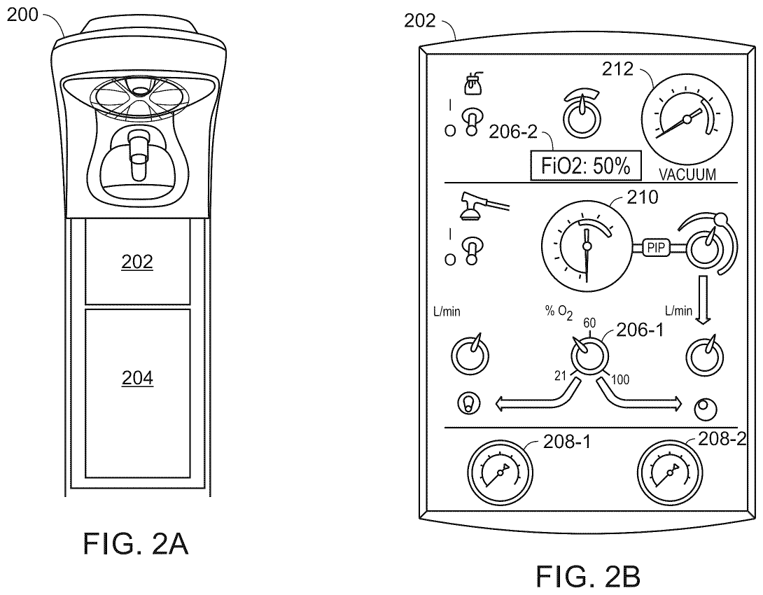

FIG. 2A is a perspective view of an example system 200 having parameter measurement for infant resuscitation devices, according to some embodiments of the present disclosure. In this example, the system 200 is an example infant warmer having a resuscitation device 202 and a platform 204. The system 200 may be similar to the infant warmer 10, described with respect to FIG. 1. Alternatively, the system 200 may be an incubation device. Additionally, the resuscitation device 202 and platform 204 may be similar to the resuscitation device 40 and platform 18, respectively.

FIG. 2B is a perspective view of the example infant resuscitation device 202 having parameter measurement, according to some embodiments of the present disclosure. In this example, the example infant resuscitation device 202 may be a pneumatic resuscitation device. In this example, the resuscitation device 202 may include an oxygen concentration control 206A (e.g., a blender control), an oxygen concentration display 206B, PEEP manometers 208-1, 208-2, peak inspiratory pressure (PIP) manometer 210, and vacuum manometer 212. The oxygen concentration control 206A may regulate how much oxygen from the gas supply 44-1 is blended with the medical grade air from gas supply 44-2. The oxygen concentration control 206-1 is referred to as a blender control because the resuscitation device 202 includes an internal component (e.g., a blender), that may blend the medical grade oxygen and air to the oxygen percentage setting indicated by the blender control. Further, the oxygen concentration control 206 includes indicators of oxygen percentage ranging from 21% to 100%. The medical grade air may include a 21% oxygen concentration. Thus, when the blender control is set to 21%, the blender of the resuscitation device 202 may limit the gas supply to the medical grade air to supply a gas containing 21% oxygen. Conversely, when the blender control is set to 100%, the blender may limit the gas supply to the medical grade oxygen to supply gas containing 100% oxygen. In this example, when the blender control is set to 50%, the blender may blend the medical grade oxygen and medical grade air to generate a gas mixture containing 50% oxygen.

According to some embodiments of the present disclosure, blender may include one or more oxygen sensors, which may be connected with the oxygen concentration display 206-2. Accordingly, the oxygen concentration display 206-2 may provide an indicator of the oxygen concentration percentage in the blender. Further, the resuscitation device 202 may power the oxygen sensors and oxygen concentration display 206-2 with power received from a universal serial bus (USB) port on the infant warmer, the battery 48, from an outlet connected to an AC power grid, and the like.

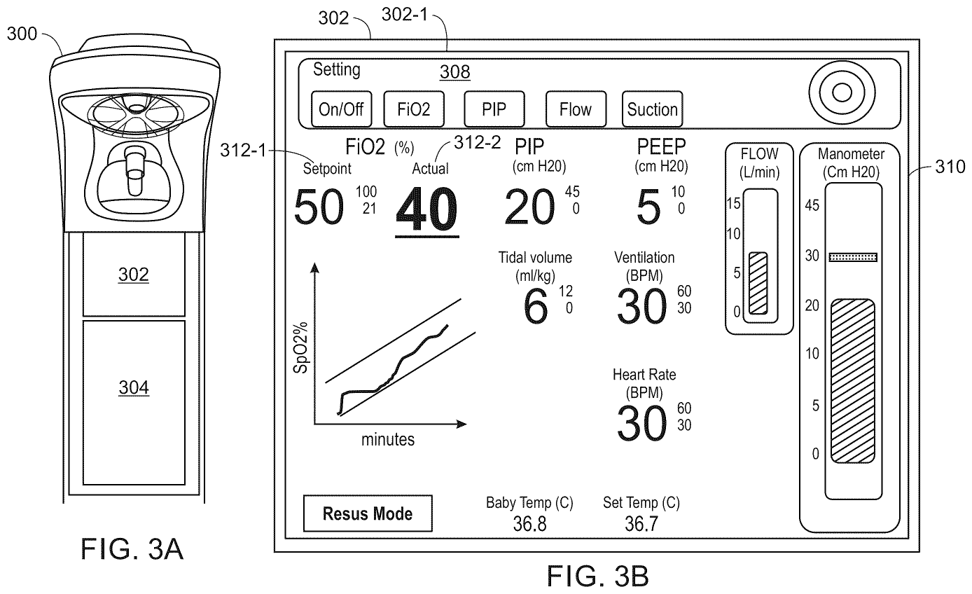

FIG. 3A is a perspective view of an example system 300 having parameter measurement for infant resuscitation devices, according to some embodiments of the present disclosure. The system 300 is an example infant warmer having an example display device 302 for a resuscitation device (not shown), and a platform 304. The system 300 and platform 304 may be respectively similar to the infant warmer 10 and platform 18, described with respect to FIG. 1.

FIG. 3B is a perspective view of the example display device 302 for a resuscitation device having parameter measurement, according to some embodiments of the present disclosure. In this example, the display device 302 includes an example touch screen user interface 302-1 for operation of the electronic resuscitation device.

The example touch screen user interface 302-1 includes controls for operating, and data describing the operation of, the electronic resuscitation device. More specifically, the touch screen user interface 302-1 includes setting controls 306. The setting controls 306 include buttons labeled: on/off, fraction of inspired oxygen (FiO₂), peak inspiratory pressure (PIP), flow, and suction. The on/off button may be useful for powering the resuscitation device on and off. The FiO₂ button may be useful for updating the oxygen concentration of the gas that the electronic resuscitation device supplies. Additionally, the PIP, flow, and suction buttons may be useful for respectively updating the the PIP, flow and suction settings of the electronic resuscitation device.

Further, the example touch screen user interface 302-1 includes FiO₂ setpoint 308-1, and FiO₂ actual 308-2. The FiO₂ setpoint 308-1 may represent the FiO2 setting, indicating the oxygen concentration percentage requested to be supplied by the electronic resuscitation device. In this example, the FiO2 setpoint 308-1 is 50%. Further, the FiO2 actual 308-2 represents the oxygen concentration percentage measured by the internal oxygen sensors of the electronic resuscitation device. In this example, the FiO2 actual 308-2 is 40%, indicating a condition where the electronic resuscitation device is not supplying the oxygen concentration percentage requested by the operator of the electronic resuscitation device. According to some embodiments of the present disclosure, the resuscitation manager 32 may provide an alert in such a scenario via a visual and/or audio signal. In this example, the FiO2 actual 308-2 is presented in bold and underlined font. Additionally, or alternatively, the resuscitation manager 32 may present the FiO2 actual 308-2 in a flashing pattern, and/or sound an audible alert, beep, or the like, to draw the attention of a healthcare provider to the operation malfunction of the electronic resuscitation device.

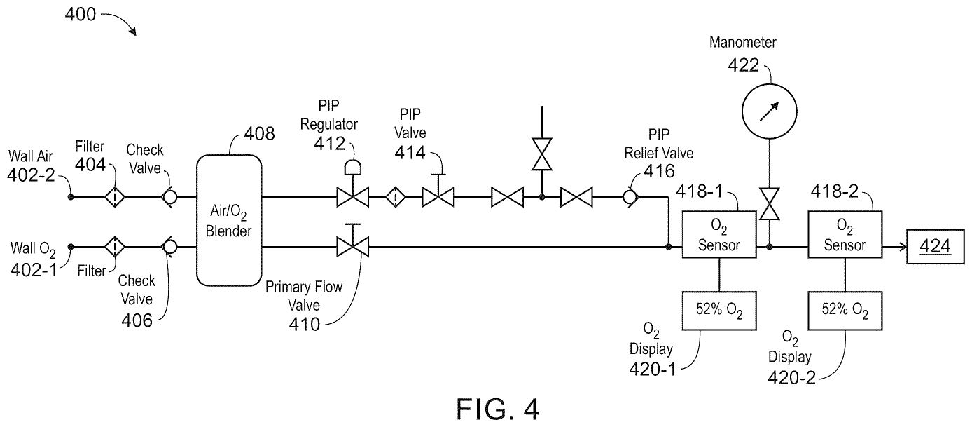

FIG. 4 is a diagram of an example respiratory circuit 400 of an infant resuscitation device having parameter measurement, according to some embodiments of the present disclosure. The example respiratory circuit 400 includes wall oxygen (O₂) 402-1, wall air 402-2, filters 404, check valves 406, air/oxygen blender 408, primary flow valve 410, PIP regulator 412, PIP valve 414, PIP relief valve 416, oxygen sensors 418-1, 418-2, oxygen displays 420-1, 420-2, manometer 422, and output to patient 424. The wall oxygen 402-1 and wall air 402-2 may be similar to the gas supply tanks 44-1, 44-2, described with respect to FIG. 1. The filters 404 may remove impurities from the air as it moves through the example respiratory circuit 400. The air/oxygen blender 408 may blend the air supplies from the wall oxygen 402-1 and wall air 402-2 based on the oxygen concentration setting of the oxygen concentration control 206-1 described with respect to FIG. 2B. The PIP regulator 412 controls the gas pressure provided to the PIP circuit. The PIP valve is user adjustable to the selected PIP pressure, which is visible on the manometer 422. The PIP relief valve allows gasses from the higher pressure primary flow gasses to flow into the PIP circuit and hence to reduce the pressure to the PIP limit before exiting to the patient. Additionally, the PIP relief valve reduces the flow rate below the one set at the primary flow valve 410. The oxygen sensors 418-1, 418-2 may measure the concentration of oxygen in the air passing through the circuit. The oxygen sensor 418-1 may measure the oxygen concentration before the air passes through the manometer 422. Further, the oxygen sensor 418-2 may measure the oxygen concentration after the air passes through the manometer 422. According to some embodiments of the present disclosure, the respiratory circuit 400 may include more than two oxygen sensors. Conversely, the example respiratory circuit 400 may merely include one oxygen sensor, which may be positioned to measure the oxygen concentration in any position on the example respiratory circuit after the air passes out of the air/oxygen blender 408. The oxygen displays 420-1, 420-2 may be connected with the oxygen sensors 418-1, 418-2, respectively, and may display the measured oxygen concentration of the connected oxygen sensor. The manometer 422 may measure the pressure of the air passing through the example respiratory circuit 400 before the air passes through the output 424 of the resuscitation device. Having multiple oxygen sensors, e.g., oxygen sensors 418-1, 418-2, providing multiple readings, e.g., on oxygen displays 420-1, 420-2, may provide additional checks against potential failures in one or both oxygen sensors 418-1, 418-2. As such, if oxygen sensors 418-1, 418-2 provide different readings, or readings with a difference that exceeds a marginal threshold, a clinician or other operator of the infant resuscitation device may determine the infant resuscitation device needs a maintenance check before the infant resuscitation device may be used safely.

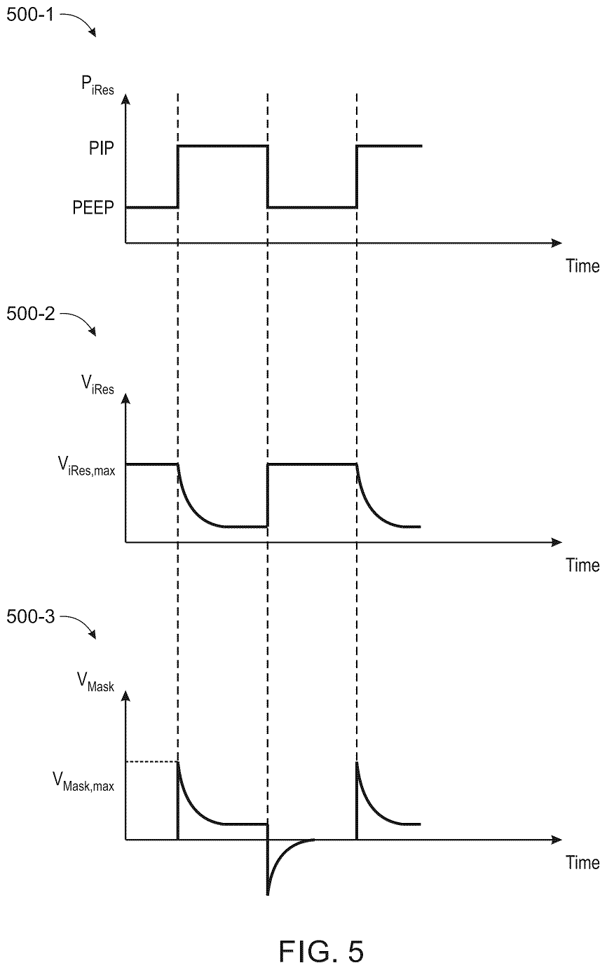

FIG. 5 is a diagram of graphs representing resuscitation device pressure 500-1, resuscitation device flow 500-2, and mask flow 500-3, over time, for a patient being administered care from an infant resuscitation device having parameter measurement, according to some embodiments of the present disclosure. Some embodiments of the present disclosure may calculate tidal volume of air that the resuscitation device provides for the patient, and the volume of any mask leak, based on the resuscitation device pressure 500-1, resuscitation device flow 500-2, and mask flow 500-3. Accordingly, some embodiments of the present disclosure may measure resuscitation device pressure 500-1 and resuscitation device flow at the exhaust of the resuscitation system. As shown, the resuscitation device pressure 500-1 varies between PIP and PEEP pressure. The dashed lines crossing the graphs indicate the changes between inflating the patient's lungs and the patient's exhale. Accordingly, the rescusitation device pressure 500-1 indicates PEEP at the lower pressure during the patient's exhale, and PIP at higher pressure while the resuscitation device inflates the patient's lungs. The variation between PEEP and PIP pressure is related to the operation at the T-piece, described with respect to FIG. 1. During the patient's exhale, (when the pressure is at PEEP), resuscitation device flow 500-2 is at the maximum flow (V_{iRes,max}). Similarly, mask flow 500-3 is at the maximum flow (V_{mask,max}). Further, when the opening at the T-Piece is covered, the resuscitation device increases the pressure from PEEP to PIP, thus inflating the patients lungs. Accordingly, as the patient's lungs are inflated, the resuscitation device flow 500-2 decreases until the the lung backpressure is equal to the PIP pressure. However, on some breaths, the resuscitation device flow 500-2 may not reach a zero flow rate. Accordingly, the amount of mask flow 500-3 at the end of the inflation cycle may represent the amount of mask leak.

Further, when the resuscitation device pressure 500-1 decreases from PIP to PEEP, the resuscitation device flow 500-2 recovers back to V_{iRes,max}. When the resuscitation device pressure 500-1 is at PEEP, the patient can exhale. Then, when the opening at the T-Piece is closed, the resuscitation device pressure 500-1 increases to PIP and inflates the patient's lungs. Towards the end of the inflation cycle, the lungs may accept a negligible amount of air. As such, all air flow measured coming out of the device may be mask leak. Thus, the V_{iRes} asymptotically approaches a constant value, which may be equal to the mask leak during the inspatory part of the cycle. Accordingly, it may be possible to correlate V_{iRes,max} and V_{mask,max}. Hence, tidal volume can be calculated for the patient.

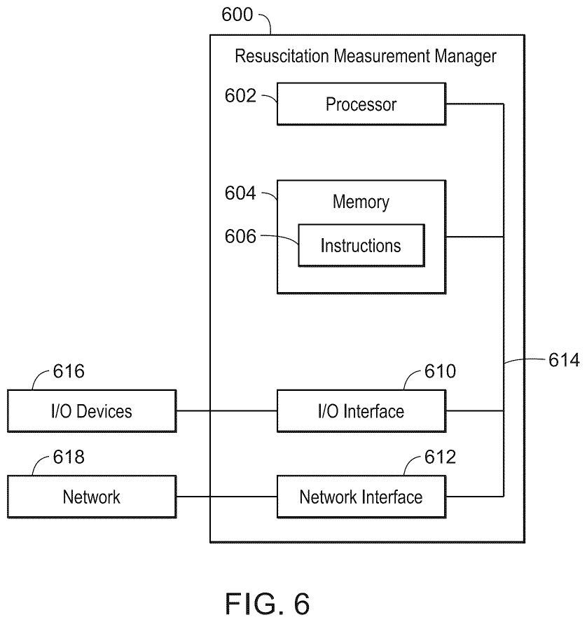

FIG. 6 is an example resuscitation measurement manager 600, according to some embodiments of the present disclosure. The example resuscitation measurement manager 600 may perform infant resuscitation parameter measurement as described with respect to FIGS. 1, 2A, 2B, 3A, 3B, 4, and 5. In this example, the resuscitation measurement manager 600 includes a processor 602, memory 604, input-output (I/O) interface 610, and network interface 612, which may be connected by an interconnect 614. The processor 602 may be a computer processing circuit (e.g., a central processing unit (CPU)) that retrieves and executes programming instructions 606 stored in the memory 604 to perform the functionality described herein. The interconnect 614 may move data, such as programming instructions, between the processor 602, memory 604, I/O interface 610, and network interface 612. The interconnect 614 may include one or more buses.

The memory 604 may be a computer memory or storage device, including volatile memory, such as a random access memory (RAM) device (e.g., static RAM, dynamic RAM, and the like), non-volatile memory, such as a hard disk drive, solid state device (SSD), removable memory cards, optical storage, flash memory devices, and the like. In some examples, the memory 604 may include volatile and non-volatile memory devices. Further, the memory 604 may store instructions 606, which may cause the processor to perform infant resuscitation parameter measurement as described with respect to FIGS. 1, 2A, 2B, 3A, 3B, 4, and 5.

Additionally, the resuscitation measurement manager 600 may be in electronic communication with I/O devices 616 through the I/O interface 610, and with a network 618 through the network interface 612. The I/O devices 616 may capture inputs and provide outputs as described herein. The network 618 may be an electronic communication network, such as a local area network, wide area network, and the like. In some examples, the network 618 may be wired, wireless (e.g., wi-fi, Bluetooth, or cellular), or some other computer communication network.

In some embodiments, the resuscitation measurement manager 600 may be a server computer or similar device without a user interface but which receives requests from other computer systems having one or more user interfaces. Further, in some embodiments, the resuscitation measurement manager 600 may be a portable computer, laptop, tablet computer, pocket computer, telephone, smart phone, or the like.

An example infant resuscitation device includes a blender control device, blender device, oxygen sensor, and display device. The blender control device selects an oxygen concentration of an air supply that the infant resuscitation device provides at an exhaust port. Additionally, the blender device generates the air supply by combining medical grade air and medical grade oxygen based on a selection of the oxygen concentration by the blender control device. Further, the oxygen sensor is disposed within a portion of a respiratory circuit of the infant resuscitation device, and generates a measure by measuring an oxygen concentration of the air supply generated by the blender device. Additionally, the display device, is disposed on an external portion of the infant resuscitation device, receives the measure from the oxygen sensor, and displays the measure of the oxygen concentration.

In an example embodiment, the first portion of the respiratory circuit is configured to receive the air supply before a manometer of the infant resuscitation device receives the air supply.

In an example embodiment, the first oxygen sensor is disposed between the blender device and the exhaust port.

In an example embodiment, the first oxygen sensor is disposed between the blender device and the manometer.

In an example embodiment, the infant resuscitation device includes a second oxygen sensor that is disposed within a second portion of the respiratory circuit of the infant resuscitation device, and is configured to generate a second measure by measuring the oxygen concentration of the air supply generated by the blender device.

In an example embodiment, the second portion of the respiratory circuit is configured to receive the air supply from a manometer of the infant resuscitation device.

In an example embodiment, the infant resuscitation device includes a circuit configured to compare the first measure to the second measure, determine if the first measure is within a predetermined threshold of the second measure, and provide an indicator for the first display to display an error message.

In an example embodiment, the infant resuscitation device includes a second display device that is disposed on a second external portion of the infant resuscitation device, and is configured to receive the second measure from the second oxygen sensor, and display the second measure of the oxygen concentration.

In an example embodiment, the infant resuscitation device includes pneumatic components configured to receive the medical grade air and the medical grade oxygen, generate the air supply by blending the medical grade air and the medical grade oxygen, and deliver the air supply under pressure at the exhaust port.

An example system includes a processor and a memory. The memory includes instructions that cause the processor to receive a set point of oxygen concentration for an infant resuscitation device, receive, from a first oxygen sensor, a first measure of oxygen concentration of air generated by a blender of the infant resuscitation device, present the first measure of oxygen concentration on an output device, compare the first measure to the set point, determine that the first measure exceeds a predetermined threshold of the set point based on the comparison, and generate, in response to the determination, an alert that the oxygen concentration is not within the predetermined threshold of the set point.

In an example embodiment, the system includes the output device, and the instructions cause the output device to perform at least one function selected from a group consisting of displaying the alert, displaying the first measure, generating an audio signal representing the alert, generating an audio message comprising the first measure.

In an example embodiment, the system includes a blender control device configured to select the set point of oxygen concentration of an air supply that the apparatus provides at an exhaust port.

In an example embodiment, the system includes the infant resuscitation device, and the infant resuscitation device includes a blender device configured to generate the air supply by combining medical grade air and medical grade oxygen based on the set point, and a first oxygen sensor that is disposed within a first portion of a respiratory circuit of the infant resuscitation device, and is configured to generate the first measure by measuring an oxygen concentration of the air supply generated by the blender device.

In an example embodiment, the first portion of the respiratory circuit is configured to receive the air supply before a manometer of the infant resuscitation device receives the air supply.

In an example embodiment, the first oxygen sensor is disposed between the blender device and the exhaust port.

In an example embodiment, the first oxygen sensor is disposed between the blender device and the manometer.

In an example embodiment, the infant resuscitation device includes a second oxygen sensor that is disposed within a second portion of the respiratory circuit of the infant resuscitation device, and is configured to generate a second measure by measuring the oxygen concentration of the air supply generated by the blender device.

In an example embodiment, the second portion of the respiratory circuit is configured to receive the air supply from a manometer of the infant resuscitation device.

In an example embodiment, the second oxygen sensor is disposed between the first oxygen sensor and the exhaust port.

In an example embodiment, the infant resuscitation device includes pneumatic components configured to receive the medical grade air and the medical grade oxygen, generate the air supply by blending the medical grade air and the medical grade oxygen, and deliver the air supply under pressure at the exhaust port.

As used herein, the term, mechanism, can encompass hardware, software, firmware, or any suitable combination thereof. In some embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as tape, hard disks, floppy disks, ands the like), optical media (such as compact discs, digital video discs, Blu-ray discs, and the like), semiconductor media (such as RAM, Flash memory, electrically programmable read only memory (EPROM), electrically erasable programmable read only memory (EEPROM), and the like), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. An infant resuscitation device, comprising:
a blender control device configured to select an oxygen concentration of an air supply that the infant resuscitation device provides at an exhaust port;
a blender device configured to generate the air supply by combining medical grade air and medical grade oxygen based on a selection of the oxygen concentration by the blender control device;
a first oxygen sensor disposed within a first portion of a respiratory circuit of the infant resuscitation device, and configured to generate a first measure by measuring the oxygen concentration of the air supply generated by the blender device; and
a first display device that is disposed on a first external portion of the infant resuscitation device, and is configured to:
receive the first measure from the first oxygen sensor; and
display the first measure of the oxygen concentration.

2. The infant resuscitation device of claim 1, wherein the first oxygen sensor is disposed between the blender device and the exhaust port.

3. The infant resuscitation device of claim 2, wherein the first portion of the respiratory circuit is configured to receive the air supply before a manometer of the infant resuscitation device receives the air supply.

4. The infant resuscitation device of claim 3, wherein the first oxygen sensor is disposed between the blender device and the manometer.

5. The infant resuscitation device of claim 1, comprising a second oxygen sensor disposed within a second portion of the respiratory circuit of the infant resuscitation device, and configured to generate a second measure by measuring the oxygen concentration of the air supply generated by the blender device.

6. The infant resuscitation device of claim 5, the second portion of the respiratory circuit being configured to receive the air supply from a manometer of the infant resuscitation device.

7. The infant resuscitation device of claim 5, comprising a circuit configured to:
compare the first measure to the second measure;
determine if the first measure is within a predetermined threshold of the second measure;
and provide an indicator for the first display device to display an error message.

8. The infant resuscitation device of claim 5, comprising a second display device that is disposed on a second external portion of the infant resuscitation device, and is configured to:
receive the second measure from the second oxygen sensor; and
display the second measure of the oxygen concentration.

9. The infant resuscitation device of claim 1, comprising a plurality of pneumatic components configured to receive the medical grade air and the medical grade oxygen, generate the air supply by blending the medical grade air and the medical grade oxygen, and deliver the air supply under pressure at the exhaust port.

10. A system, comprising:
a processor; and
a memory comprising instructions configured to cause the processor to:
receive a set point of oxygen concentration for an infant resuscitation device;
receive, from a first oxygen sensor, a first measure of oxygen concentration of air generated by a blender of the infant resuscitation device;
present the first measure of oxygen concentration on an output device;
compare the first measure to the set point;
determine that the first measure exceeds a predetermined threshold of the set point based on the comparison; and
generate, in response to the determination, an alert that the first measure is not within the predetermined threshold of the set point.

11. The system of claim 10, comprising the output device, the instructions being configured to cause the output device to perform at least one function selected from a group consisting of displaying the alert, displaying the first measure, generating an audio signal representing the alert, generating an audio message comprising the first measure.

12. The system of claim 10, comprising a blender control device configured to select the set point of oxygen concentration of an air supply that the infant resuscitation device provides at an exhaust port.

13. The system of claim 12, comprising the infant resuscitation device, the infant resuscitation device comprising:
a blender device configured to generate the air supply by combining a medical grade air and a medical grade oxygen based on the set point; and
the first oxygen sensor, wherein the first oxygen sensor is disposed within a first portion of a respiratory circuit of the infant resuscitation device, and configured to generate the first measure by measuring an oxygen concentration of the air supply.

14. The system of claim 13, wherein the first oxygen sensor is disposed between the blender device and the exhaust port.

15. The system of claim 14, wherein the first portion of the respiratory circuit is configured to receive the air supply before a manometer of the infant resuscitation device receives the air supply.

16. The system of claim 15, wherein the first oxygen sensor is disposed between the blender device and the manometer.

17. The system of claim 13, the infant resuscitation device comprising a second oxygen sensor disposed within a second portion of the respiratory circuit of the infant resuscitation device, and configured to generate a second measure by measuring the oxygen concentration of the air supply generated by the blender device.

18. The system of claim 17, the second portion of the respiratory circuit being configured to receive the air supply from a manometer of the infant resuscitation device.

19. The system of claim 18, wherein the second oxygen sensor is disposed between the first oxygen sensor and the exhaust port.

20. The system of claim 13, the infant resuscitation device comprising a plurality of pneumatic components configured to receive the medical grade air and the medical grade oxygen, generate the air supply by blending the medical grade air and the medical grade oxygen, and deliver the air supply under pressure at the exhaust port.
